(19) United States
(12) Patent Application Publication
(10) Pub. No.: US 2013/0190276 A1
(43) Pub. Date: Jul. 25, 2013

(54) USE OF 5-H-DIBENZ/B,F/AZEPINE-5-CARBOXAMIDE DERIVATIVES IN THE TREATMENT OF NEUROPATHIC PAIN AND NEUROLOGICAL DISORDERS

(71) Applicant: BIAL - PORTELA & CA, S.A., S. Mamede do Coronado (PT)

(72) Inventor: Patricio Manuel Vieira Araujo Soares da Silva, Porto (PT)

(73) Assignee: BIAL - PORTELA & CA, S.A., S. Mamede do Coronado (PT)

(21) Appl. No.: 13/790,925

(22) Filed: Mar. 8, 2013

Related U.S. Application Data
(63) Continuation of application No. 13/342,777, filed on Jan. 3, 2012.
Application No. 13/342,777 is a continuation of application No. 12/279,027, filed on Aug. 11, 2008, now abandoned.

(30) Foreign Application Priority Data
Feb. 14, 2006 (GB) 0603008.4

(51) Int. Cl. A61K 31/55; A61K 45/06

(52) U.S. Cl. CPC A61K 31/55; A61K 45/06
USPC 514/161; 514/217

(57) ABSTRACT

This invention relates to the use of 5H-dibenz/b,f/azepine-5-carboxamide derivatives in the manufacture of various medicaments for treating neuropathic pain and for treating neurological disorders which involve both motor impairment and neuropathic pain.

This invention relates to the use of 5H-dibenz/b,f/azepine-5-carboxamide derivatives in the manufacture of various medicaments for treating neuropathic pain and for treating neurological disorders which involve both motor impairment and neuropathic pain.

Eslicarbazepine acetate (ESL, S-(−)-10-acetoxy-10,11-dihydro-5H-dibenz/b,f/azepine-5-carboxamide; also known as BIA 2-093) is a new voltage-gated sodium channel (VGSC) blocker that shares with carbamazepine (CBZ) the dibenzazepine nucleus bearing the 5-carboxamide substituent, but is structurally different at the 10,11-position (see BENES, J., PARADA, A., FIGUEIREDO, A. A., ALVES, P. C., FREITAS, A. P., LEARMONTH, D. A., CUNHA, R. A., GARRETT, J. & SOARES-DA-SILVA, P, (1999), ‘Anti-convulsant and sodium channel-blocking properties of novel 10,11-dihydro-5H-dibenz[b,f]azepine-5-carboxamide derivatives’, J Med Chem, 42, 2582-2587).

This molecular variation results in differences in metabolism, namely by preventing the formation of toxic epoxide metabolites, such as carbamazepine-10,11 epoxide, and unnecessary production of enantiomers or diastereoisomers of metabolites and conjugates (see HAINZL, D., PARADA, A. & SOARES-DA-SILVA, P. (2001), ‘Metabolism of two new antiepileptic drugs and their principal metabolites S(+)—and R(−)-10,11-dihydro-10-hydroxy carbamazepine’, Epilepsy Res, 44, 197-206), without losing pharmacological activity (see the above Benes reference). ESL was shown to be an effective anticonvulsant in rats and mice and to exert protecting effects against maximal electroshock seizure (MES) and a variety of convulsant agents. In the rat model, ESL was found to be particularly active against MES-induced seizures with anticonvulsant potency similar to that for CBZ, but more potent than oxcarbazepine (OXC, see the above Benes reference).

Mechanistically, ESL appears not to interfere with receptors for benzodiazepines, GABA and glutamate, but behaves as a potent blocker of VGSC by competitively interacting with site 2 of the inactivated state of the channel (see AMBROSIO, A. F., SILVA, A. P., MALVA, J. O., SOARES-DA-SILVA, P., CARVALHO, A. P. & CARVALHO, C. M. (2001), ‘Inhibition of glutamate release by BIA 2-093 and BIA 2-024, two novel derivatives of carbamazepine, due to blockade of sodium but not calcium channels’, Biochem Pharmacol, 61, 1271-1275; AMBROSIO, A. F., SOARES-DA-SILVA, P., CARVALHO, C. M. & CARVALHO, A. P. (2002), ‘Mechanisms of action of carbamazepine and its derivatives, oxcarbazepine, BIA 2-093, and BIA 2-024’, Neurochem Res, 27, 121-130; and the above Benes reference). Its affinity for this state of the channel was similar to that of CBZ, while the affinity for the resting state of the channel was about 3-fold lower than that of CBZ. This profile may suggest an enhanced inhibitory selectivity of ESL for rapidly firing neurones over those displaying normal activity (see BONIFACIO, M. J., SHERIDAN, R. D., PARADA, A., CUNHA, R. A., PATMORE, L. & SOARES-DA-SILVA, P. (2001), ‘Interaction of the novel anticonvulsant, BIA 2-093, with voltage-gated sodium channels: comparison with carbamazepine’, Epilepsia, 42, 600-608).

The human metabolite of oxcarbazepine is also known as licarbazepine and exhibits comparable antiepileptic activity to the parent drug (Benes et. al., 1999; Schutz et al., 1986). Use of this metabolite as an antiepileptic drug was described, but it is not used in practice. It was also found that this metabolite which is chiral in nature, is not formed in a totally stereoselective manner in humans, and S-licarbazepine (S-Lic) and R-licarbazepine (R-Lic) are formed in proportions of approximately 80% to 20%, respectively. Exact proportions of those enantiomers are moreover subject-dependent. They are metabolised further at different rates and form different enantiomers and numerous diastereoisomers of metabolites and conjugates, with possibly widely different pharmacodynamic and pharmacokinetic behaviour, as well as side effects.

From a mechanistic point of view, the anticonvulsant effects of oxcarbazepine are considered to result from blockade of voltage-gated sodium channels (VGSC) by competitively interacting with site 2 of the inactivated state of the channel (Ambrosio et al., 2001; Ambrosio et al., 2002; Benes et al., 1999). However, despite evidence suggesting that the therapeutic effects of oxcarbazepine in humans are related to the effects of its main metabolite (Baruzzi et al., 1994; Leppik, 1994; Lloyd et al., 1994; May et al., 1996), the interaction of S-licarbazepine and R-licarbazepine has not been evaluated in detail.

According to a first aspect of the present invention, there is provided the use of a 5H-dibenz/b,f/azepine-5-carboxamide derivative selected from eslicarbazepine acetate, R-licarbazepine acetate or a mixture of eslicarbazepine acetate and R-licarbazepine acetate in any proportion in the manufacture of a medicament for treating neuropathic pain.

In an embodiment, the 5H-dibenz/b,f/azepine-5-carboxamide derivative is the racemate of eslicarbazepine acetate and R-licarbazepine acetate.

According to a second aspect of the present invention, there is provided the use of a 5H-dibenz/b,f/azepine-5-carboxamide derivative selected from eslicarbazepine acetate, R-licarbazepine acetate or a mixture of eslicarbazepine acetate and R-licarbazepine acetate in any proportion in combination with a nonselective COX inhibitor selected from: acetylsalicylic acid, sodium salicylate, choline, magnesium trisalicylate, salsalate, diflunisal, sulfasalazine, olsalazine, or combinations thereof; acetaminophen; indometahcin, sulindac, or combinations thereof; tolmetin, diclofenac, ketorelac, or combinations thereof; ibuprofen, naproxen, flurbiprofen, ketoprofen, fenoprofen, oxaprozin, or combinations thereof; mephenamic acid, meclofenamic acid, or combinations thereof; Piroxicam, meloxicam, or combinations thereof; and nabumetone, a selective COX inhibitor selected from: rofecoxib, celecoxib, etoricoxib, parecoxib, valdecoxib, lumiracoxib, cimicoxib, or combinations thereof; Etodolac; and Nimesulide, opioid receptor agonists selected from Morphine, methadone, etorphine, codeine, hydrocodone, oxycodone, tramadol, levorphanol, meperidine, propoxyphene, fentanyl, sufentanil, alfentanil, remifentanil, and combinations thereof, and/or opioid receptor partial agonists selected from pentazocine, butorphanol, buprenorphine and combinations thereof in the manufacture of a medicament for treating neuropathic pain.

In an embodiment, the 5H-dibenz/b,f/azepine-5-carboxamide derivative is eslicarbazepine acetate.

In an embodiment, the 5H-dibenz/b,f/azepine-5-carboxamide derivative is R-licarbazepine acetate.

In an embodiment, the 5H-dibenz/b,f/azepine-5-carboxamide derivative is a mixture of eslicarbazepine acetate and R-licarbazepine acetate in any proportion.

In an embodiment, the 5H-dibenz/b,f/azepine-5-carboxamide derivative is the racemate of eslicarbazepine acetate and R-licarbazepine acetate.

According to a third aspect of the present invention, there is provided the use of a 5H-dibenz/b,f/azepine-5-carboxamide derivative selected from eslicarbazepine acetate, R-licarbazepine acetate, a mixture of eslicarbazepine acetate and R-licarbazepine acetate in any proportion, S-licarbazepine, R-licarbazepine, a mixture of S-licarbazepine and R-licarbazepine in any proportion, oxcarbazepine and carbamazepine in the manufacture of a medicament for treating neurological disorders which involve both motor impairment and neuropathic pain.

In an embodiment, the 5H-dibenz/b,f/azepine-5-carboxamide derivative is the racemate of eslicarbazepine acetate and R-licarbazepine acetate.

In an embodiment, the 5H-dibenz/b,f/azepine-5-carboxamide derivative is the racemate of S-licarbazepine and R-licarbazepine.

According to a fourth aspect of the present invention, there is provided the use of a 5H-dibenz/b,f/azepine-5-carboxamide derivative selected from eslicarbazepine acetate, R-licarbazepine acetate, a mixture of eslicarbazepine acetate and R-licarbazepine acetate in any proportion, S-licarbazepine, R-licarbazepine, a mixture of S-licarbazepine and R-licarbazepine in any proportion, oxcarbazepine and carbamazepine in combination with a nonselective COX inhibitor selected from: acetyl-salicylic acid, sodium salicylate, choline, magnesium trisalicylate, salsalate, diflunisal, sulfasalazine, olsalazine, or combinations thereof; acetaminophen; indometahcin, sulindac, or combinations thereof; tolmetin, diclofenac, ketorelac, or combinations thereof; ibuprofen, naproxen, flurbiprofen, ketoprofen, fenoprofen, oxaprozin, or combinations thereof; mephenamic acid, meclofenamic acid, or combinations thereof; Piroxicam, meloxicam, or combinations thereof; and nabumetone, a selective COX inhibitor selected from: rofecoxib, celecoxib, etoricoxib, parecoxib, valdecoxib, lumiracoxib, cimicoxib, or combinations thereof; Etodolac; and Nimesulide, opioid receptor agonists selected from Morphine, methadone, etorphine, codeine, hydrocodone, oxycodone, tramadol, levorphanol, meperidine, propoxyphene, fentanyl, sufentanil, alfentanil, remifentanil, and combinations thereof, and/or opioid receptor partial agonists selected from pentazocine, butorphanol, buprenorphine and combinations thereof in the manufacture of a medicament for treating neurological disorders which involve both motor impairment and neuropathic pain.

In an embodiment, the 5H-dibenz/b,f/azepine-5-carboxamide derivative is eslicarbazepine acetate.

In an embodiment, the 5H-dibenz/b,f/azepine-5-carboxamide derivative is R-licarbazepine acetate.

In an embodiment, the 5H-dibenz/b,f/azepine-5-carboxamide derivative is a mixture of eslicarbazepine acetate and R-licarbazepine acetate in any proportion.

In an embodiment, the 5H-dibenz/b,f/azepine-5-carboxamide derivative is the racemate of eslicarbazepine acetate and R-licarbazepine acetate.

In an embodiment, the 5H-dibenz/b,f/azepine-5-carboxamide derivative is S-licarbazepine.

In an embodiment, the 5H-dibenz/b,f/azepine-5-carboxamide derivative is R-licarbazepine.

In an embodiment, the 5H-dibenz/b,f/azepine-5-carboxamide derivative is a mixture of S-licarbazepine and R-licarbazepine in any proportion.

In an embodiment, the 5H-dibenz/b,f/azepine-5-carboxamide derivative is the racemate of S-licarbazepine and R-licarbazepine.

In an embodiment, the 5H-dibenz/b,f/azepine-5-carboxamide derivative is oxcarbazepine.

In an embodiment; the 5H-dibenz/b,f/azepine-5-carboxamide derivative is carbamazepine.

In an embodiment, the disorder is selected from polyneuropathies, multiple sclerosis, Parkinson disease, CNS diseases (caused by vascular, tumoral and inflammatory processes) with de-efferentiation, motor neuron disease, progressive supranuclear palsy, multiple system atrophy, corticobasal degeneration, spinocerebellar ataxia, cervical myelopathy, spinal cord injury and radicular avulsion.

According to a fifth aspect of the present invention, there is provided a method of treating neuropathic pain comprising administering to a subject in need thereof a therapeutically effective amount of a 5H-dibenz/b,f/azepine-5-carboxamide derivative selected from eslicarbazepine acetate, R-licarbazepine acetate or a mixture of eslicarbazepine acetate and R-licarbazepine acetate in any proportion.

According to a sixth aspect of the present invention, there is provided a method of treating neurological disorders which involve both motor impairment and neuropathic pain comprising administering to a subject in need thereof a therapeutically effective amount of a 5H-dibenz/b,f/azepine-5-carboxamide derivative selected from eslicarbazepine acetate, R-licarbazepine acetate, mixtures of eslicarbazepine acetate and R-licarbazepine acetate in any proportion, S-licarbazepine, R-licarbazepine, mixtures of S-licarbazepine and R-licarbazepine in any proportion, oxcarbazepine and carbamazepine.

Neuropathic pain and neuropathic pain related disorders include trigeminal neuralgia, phantom pain, diabetic neuropathy and postherpetic neuralgia.

Another neurological deficit is motor impairment. We have surprisingly found that ESL, R-Lic acetate, S-Lic and R-Lic produce considerably less motor impairment, and are more effective in treating neuropathic pain, than CBZ and OXC. Thus, ESL, R-Lic acetate, a mixture of ESL and R-Lic acetate in any proportion, S-Lic, R-Lic, and a mixture of S-Lic and R-Lic in any proportion confer improved efficacy upon the treatment of neurological disorders which involve both neuropathic pain and motor impairment. The racemate of ESL and R-Lic acetate is an example of a mixture of ESL and R-Lic acetate in any proportion. The racemate of S-Lic and R-Lic is an example of a mixture of S-Lic and R-Lic in any proportion.

We have found that ESL is particularly advantageous in the treatment of neurological disorders which involve both motor impairment and neuropathic pain. Neurological disorders which involve both neuropathic pain and motor impairment include polyneuropathies, multiple sclerosis, Parkinson disease, CNS diseases (caused by vascular, tumoral and inflammatory processes) with de-eferentiation, motor neuron disease, progressive supranuclear palsy, multiple system atrophy, corticobasal de-generation, spinocerebellar ataxia, cervical myelopathy, spinal cord injury and radicular avulsion.

As used herein the expression neurological disorders which involve both motor impairment and neuropathic pain, and like expressions, includes ‘neurological disorders which cause both motor impairment and neuropathic pain’.

As used herein, the term treatment and variations such as ‘treat’ or ‘treating’ refer to any regime that can benefit a human or non-human animal. The treatment may be in respect of an existing condition or may be prophylactic (preventative treatment). Treatment may include curative, alleviation or prophylactic effects.

Another unexpected advantage of the 5H-dibenz/b,f/azepine-5-carboxamide derivatives of the present invention is that they do not induce too much sedation as a side-effect. This is particularly the case when the following 5H-dibenz/b,f/azepine-5-carboxamide derivatives are used in the medicament: ESL, R-licarbazepine acetate, mixtures of ESL and R-licarbazepine acetate in any proportion (including the racemate of ESL and R-licarbazepine acetate), R-Lic, S-Lic, and mixtures of S-Lic and R-Lic in any proportion (including the racemate of S-Lic and R-Lic).

It has also been surprisingly found that the degree of interaction of S-licarbazepine and R-licarbazepine with site 2 in voltage-gated sodium channels is approximately 2.5 times less than that for oxcarbazepine, indicating that the analgesic effects of S-licarbazepine and R-licarbazepine, or a mixture thereof, may be not due, as for ox carbazepine, to the blockade of voltage-gated sodium channels.

Reference is made to the accompanying Figures in which:

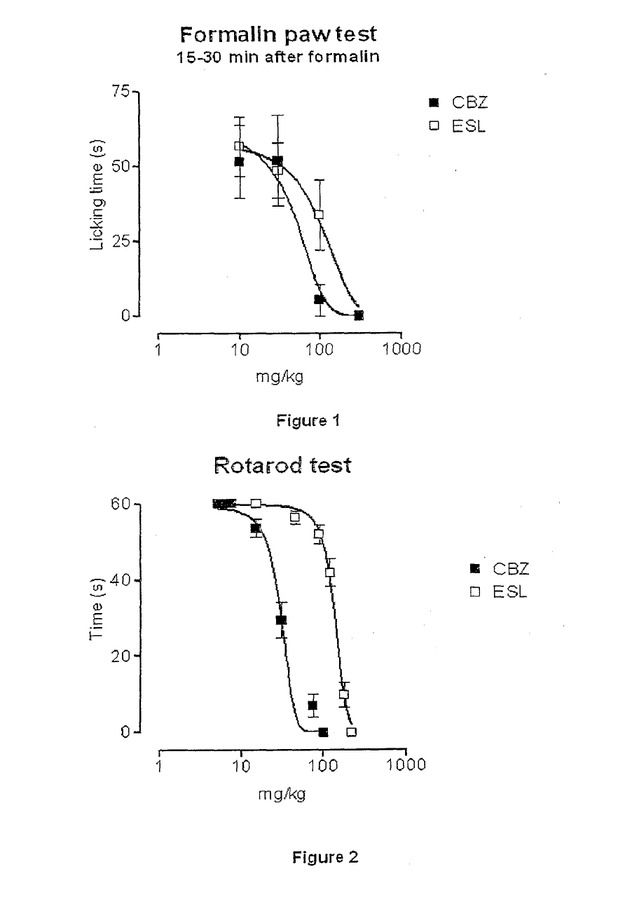

FIG. 1—Effect of eslicarbazepine acetate (ESL) and carbamazepine (CBZ) on licking time in the formalin paw test in mice. Symbols are means of 10 animals per group; vertical lines indicate S.E.M. values.

FIG. 2—Effect of eslicarbazepine acetate (ESL) and carbamazepine (CBZ) on time spent in the rotating rod. Symbols are means of 15-30 animals per group; vertical lines indicate S.E.M. values.

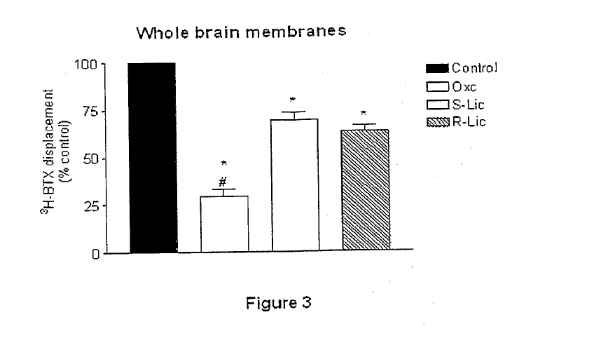

FIG. 3—Effect of oxcarbazepine (OXC), S-licarbazepine (S-Lic) and R-licarbazepine (R-Lic) on displacement of [3H]-batrachotoxinin A 20-alpha-benzoate ([3H]-BTX) binding site in whole brain membranes. Symbols are means of 4-5 independent experiments per group; vertical lines indicate S.E.M. values. Significantly different from control values (* P<0.05) and values for S-Lic (# P<0.05) and R-Lic (# P<0.05).

The invention will now be described with reference to the following non-limiting examples.

### Treatment of Neuropathic Pain

It is known that neuropathic pain can be measured by the formalin paw licking test, and motor impairment can be measured by the rotarod test. Both tests were carried out on ESL, CBZ, R-Lic and OXC, as now detailed.

Materials and Methods

Formalin Paw Test

The method, which detects analgesic/anti-inflammatory activity, follows that described by Wheeler-Aceto et al (see WHEELER-ACETO, H. & A., C. (1991), ‘Standardization of the rat paw formalin test for the evaluation of analgesics’, Psychopharmacology, 104, 35-44). Mice (NMRI) were given an intraplantar injection of 5% formalin (25 μl) into the posterior left paw. This treatment induced paw licking in control animals. The time spent licking was counted for 15 minutes, beginning 15minutes after injection of formalin. 10 mice were studied per group. The test was performed blind. ESL and CBZ were tested at the doses of 10, 30, 100 and 300 mg/kg p.o., and OXC and R-Lic were tested at the doses of 100 and 300 mg/kg p.o., administered 60 minutes before the test (i.e. 45 minutes before formalin), and compared with a vehicle control group in each experiment. Morphine (64 mg/kg p.o.), administered under the same experimental conditions, will be used as reference substance.

Rotarod Test

A normal mouse can maintain its equilibrium for long periods in the rotating rod. Mice were examined for motor toxicity in the rotating rod apparatus (Accelerator Rota-Rod [Jones & Roberts] 7650; Ugo Basile). The motor performance of naive mice (male Charles River , weighing 30 to 35 g) was evaluated 15 min after the administration of the compounds to be tested. Animals were placed on the rotating rod at a speed of 15 r.p.m.. In a drug-treated mouse the neurological deficit is indicated by the inability of the animal to maintain equilibrium for 1 min in each of three trials. ESL, CBZ, OXC and R-Lic were dissolved in dimethyl sulfoxide (DMSO) (2 ml/kg) and given intraperitoneally (see ROGAWSKI, M. A., YAMAGUCHI, S., JONES, S. M., RICE, K. C., THURKAUF, A. & MONN, J. A. (1991). Anticonvulsant activity of the low-affinity uncompetitive N-methyl-D-aspartate antagonist (+)-5-aminocarbonyl-10,11-dihydro-5H-dibenzo[a,d]cyclohepten-5,10-imine (ADCI): comparison with the structural analogs dizocilpine (MK-801) and carbamazepine. J Pharmacol Exp Ther, 259, 30-37).

Results

ESL and CBZ

Licking time (in seconds) in vehicle-treated mice was 81.0±13.8 (n=10). Both ESL and CBZ reduced licking time in a dose-dependent manner (FIG. 1) with ED₅₀ values (in mg/kg) of 69.7 and 38.2, respectively. At 300 mg/kg both compounds abolished licking in the formalin test.

Sedation was observed in 1/10 and 10/10 mice given 300 mg/kg ESL and 300 mg/kg CBZ, respectively. Morphine (64 mg/kg), administered under the same experimental conditions, completely inhibited licking (−100%, p<0.01).

The administration of increasing doses of ESL and CBZ intraperitoneally, conferred a dose-dependent motor impairment in the rotarod test, which was considerably more marked for the latter. FIG. 2 shows the dose-response curve in the rotarod test with a ED₅₀ of 139.1 and 29.7 mg/kg, respectively, for ESL and CBZ.

Considering the Efficacy-Risk (Motor) Index (formalin paw test licking tkne/ED₅₀ in the rotarod test) as a measure of therapeutic tolerability, these data indicate that ESL is better tolerated than CBZ.

Formalin paw test licking time (s) at 100 mg/kg and rotarod test ED₅₀ values (in mg/kg) were measured for CBZ, ESL, OXC, and R-Lic to compare the efficacy-risk (motor) indexes (Formalin paw test licking time (s) at 100 mg/kg/ Rotarod test ED₅₀ values (in mg/kg)) and the efficacy-risk (sedation) indexes (Formalin paw test licking time (s) at 100 mg/kg/Sedation (%) at 300 mg/kg) for all the compounds. From these values, the overall efficacy-risk index (motor x sedation) was calculated (Table 1) i.e. Efficacy-Risk (Motor) Index/% sedation.

TABLE 1
Efficacy-risk (motor) index, efficacy-risk (sedation) index and
efficacy-risk (motor × sedation) indexes

| | CBZ | ESL | Oxc | R-Lic |
|---|---|---|---|---|
| Formalin paw | 5.3 | 33.6 | 1 | 36.7 |
| test licking time (s) | | | | |
| at 100 mg/kg | | | | |
| Rotarod test | 29.7 | 139.1 | 50.2 | 97.4 |
| ED50 values (in | | | | |
| mg/kg) | | | | |
| Efficacy-Risk | 0.18 | 0.24 | 0.02 | 0.38 |
| (motor) Index | | | | |
| Safety margin | 9.0 | 12.1 | 1.0 | 18.9 |
| over ox- | | | | |
| carbazepine | | | | |
| Safety margin | 1 | 1.4 | 0.1 | 2.1 |
| over car- | | | | |
| bamazepine | | | | |
| Sedation (%) at | 100 | 10 | 90 | 20 |
| 300 mg/kg | | | | |
| Efficacy-Risk | 0.05 | 3.36 | 0.01 | 1.84 |
| (sedation) | | | | |
| Index | | | | |
| Safety margin | 4.8 | 302.4 | 1.0 | 165.2 |
| over ox- | | | | |
| carbazepine | | | | |
| Safety margin | 1.0 | 63.4 | 0.2 | 34.6 |
| over car- | | | | |
| bamazepine | | | | |
| Efficacy-Risk | 0.002 | 0.024 | 0.000 | 0.019 |
| (motor | | | | |
| impairment × | | | | |
| sedation) Index | | | | |
| Safety margin | 8.1 | 109.1 | 1.0 | 85.1 |
| over ox- | | | | |
| carbazepine | | | | |
| Safety margin | 1.0 | 13.5 | 0.1 | 10.6 |
| over car- | | | | |
| bamazepine | | | | |

Discussion

(1) ESL and CBZ

As shown in FIG. 1, CBZ behaved slightly more potently than ESL on the formalin paw test. In the Rotarod test, CBZ was found to produce in lower doses considerable motor impairment, which did not occur with ESL. The Efficacy-Risk (motor) Index for ESL was 1.4-fold that observed for CBZ, which indicates that ESL confers improved overall efficacy upon the treatment of painful conditions over CBZ. Without wishing to be bound by theory, it is thought that this surprising effect may relate to the selectivity of ESL for rapidly firing neurones over those displaying normal activity.

R-Lic and OXC

In the Rotarod test, oxcarbazepine was found to produce in lower doses considerable motor impairment, which did not occur with R-licarbazepine. The Efficacy-Risk (motor) Index for R-licarbazepine was 18.9-fold that observed for oxcarbazepine, which indicates that R-licarbazepine. confers improved efficacy upon the treatment of painful conditions over oxcarbazepine. Without wishing to be bound by theory, it is thought that this surprising effect may relate to the reduced affinity of R-licarbazepine for voltage-gated sodium channels.

ESL, CBZ, R-Lic and OXC

When considering treatment of neuropathic pain and reduction of motor impairment, R-Lic is particularly efficacious in treating neuropathic pain and limiting motor impairment. ESL is also efficacious, but to a lesser extent. Both are more efficaceous than OXC and CBZ.

When considering treatment of neuropathic pain without the inducement of sedation as a side-effect, ESL is the most effective. R-Lic is also efficacious in this regard, but less so than ESL. Both are more efficacious than OXC and CBZ.

The overall situation when considering treating neuropathic pain, without the inducement of sedation and whilst reducing motor impairment, is that ESL is the most efficacious.

The metabolism of oxcarbazepine in mice (Hainzl et al., 2001) is identical to that described in humans (Almeida et al., 2005) and for such a reason, mice should be considered the most relevant species to evaluate the benefits and risks involving the use of oxcarbazepine. Of great relevance is the observation that mice when administered with S-licarbazepine or R-licarbazepine do not convert these materials back to oxcarbazepine (Hainzl et al., 2001). In contrast, the administration of oxcarbazepine to mice results, as in humans, in conversion of oxcarbazepine to a mixture of S- and R-licarbazepine, also known as MHD. This conversion of oxcarbazepine to S- and R-licarbazepine is not complete, and levels of oxcarbazepine in the circulation and brain are measurable for a considerable period of time. Without wishing to be bound by theory, it is thought that the presence of oxcarbazepine itself in the brain is the cause for its reduced tolerability in treating pain.

Neuropathic pain is caused by damage to somatosensible afferent nerve fibres in the peripheral or central nervous system. Often, the pain cannot be satisfactorily treated with nonsteroidal anti-inflammatory drugs. Dependent on the underlying mechanism it is of therapeutic interest to consider the combined administration of ESL, S-licarbazepine, R-licarbazepine or mixtures thereof that decrease neuronal firing, and drugs acting at different levels of the aforementioned systems. These include the combined administration of ESL, R-Lic acetate, mixtures of ESL and R-Lic acetate in any proportion (including the racemate of ESL and R-Lic acetate), S-Lic, R-Lic, mixtures of S-Lic and R-Lic in any proportion (including the racemate of S-Lic and R-Lic), OXC and CBZ or mixtures thereof and one or more of the drugs selected from one or more of the classes of drugs listed in Table 2.

TABLE 2
ESL and Analgesic Drug Combinations of Therapeutic Interest

| | |
|---|---|
| Nonselective COX | Salicylic acid derivatives (acetylsalicylic |
| inhibitors | acid, sodium salicylate, choline |
| | magnesium trisalicylate, salsalate, |
| | diflunisal, sulfasalazine and/or olsalazine) |
| | Para-aminophenol derivatives |
| | (acetaminophen) |
| | Indole and indene acetic acids |
| | (indometahcin and/or sulindac) |
| | Heteroaryl acetic acids (tolmetin, |
| | diclofenac and/or ketorelac) |
| | Arylpropionic acids (ibuprofen, naproxen, |
| | flurbiprofen, ketoprofen, fenoprofen and/ |
| | or oxaprozin) |
| | Anthranilic acids (mephenamic acid and/ |
| | or meclofenamic acid) |
| | Enolic acids (Piroxicam and/or |
| | meloxicam) |
| | Alkanones (nabumetone) |
| Selective COX inhibitors | Diaryl-substituted derivatives (rofecoxib, |
| | celecoxib, etoricoxib, parecoxib, |
| | valdecoxib, lumiracoxib and/or cimicoxib) |
| | Indole acetic acids (Etodolac) |
| | Sulfonanilides (Nimesulide) |
| Opioid receptor agonists | Morphine, methadone, etorphine, codeine, |
| | hydrocodone, oxycodone, tramadol, |
| | levorphanol, meperidine, propoxyphene, |
| | fentanyl, sufentanil, alfentanil and/or |
| | remifentanil |
| Opioid receptor partial | pentazocine,butorphanol and/or |
| agonists | buprenorphine |

### Blockage of Voltage-Sensitive Sodium Channels by OXC, S-Lic and R-Lic

Materials and Methods

[3H]BTX Binding

Blockade of voltage-sensitive sodium channels was studied by investigating [3H] batrachotoxinin A 20-R-benzoate ([3H]BTX) displacement binding to whole brain membranes. Animals were decapitated and their brains quickly removed. Membrane preparation and binding assays were performed essentially as previously described (Shimidzu et al., 1997). Brains (without cerebellum) were homogenised in 10 vol 0.32 M sucrose, 1 mM EDTA, 1 mg/ml bovine serum albumin (BSA), 5 mM HEPES/TRIS pH 7.4 with a Teflon homogeniser (8 strokes at 400 r.p.m). After a 10 min centrifugation at 1,000 g the supernatants were centrifuged for 20 min at 39,000 g and pellets were homogenised with 20 vol or 40 vol Na+-free buffer, respectively for [3H]-batrachotoxinin A 20-alpha-benzoate ([3H]-BTX) binding assays. Na+-free buffer had the following composition (in mM): 130 choline chloride, 0.8 MgSO4, 5.4 KCl, 5.5 D-glucose, 50 HEPES/TRIS, pH 7.4. The homogenate was centrifuged for 20 min at 39,000 g and the resultant pellets were resuspended in Na+-free buffer. Protein concentration in membrane preparations was determined with BioRad Protein Assay (BioRad) using a standard curve of BSA (50-250 μg/ml). In [3H]-BTX binding assay experiments membrane preparations (200 μg protein) were incubated for 1 h at 37° C. with 10 nM (inhibition experiments) or 1-200 nM (saturation experiments) [3H]-BTX in Na+-free buffer containing 2 μM scorpion toxin, 1 μM tetrodotoxin and 1 mg/ml BSA in 96-well EIA/RIA plates (COSTAR). In inhibition experiments the reaction buffer contained also 3-1000 μM of test drugs. Nonspecific binding was determined in the presence of 300 μM veratridine. Nonspecific binding was 26±2% of total binding at 10 nM [3H-BTX]. After incubation the reaction was terminated by vacuum filtration (Brandel 96 harvester) through glassfiber filtermats (Wallac). Filters were washed 3times with ice-cold wash buffer (1 mg/ml BSA, 130 mM choline chloride, 0.8 mM MgSO4, 1.8 mM CACl2, 5 mM HEPES/TRIS pH 7.4). Filtermats were dried, impregnated with MeltiLex A scintillation mixture (Wallac), inserted into plastic sample bags (Wallac) and radioactivity determined in a Microbeta 1224-510 counter (Wallac).

Results

The improved performance of R-licarbazepine over oxcarbazepine in treating neuropathic pain is inversely correlated with the potency of R-Lic upon the interaction of site 2 in voltage-gated sodium channels as indicated by their reduced ability to displaced [3H]-batrachotoxinin A 20-alpha-benzoate ([3H]-BTX) from its binding site in whole brain membranes (FIG. 3). Thus, without wishing to be bound by theory, the most likely explanation is that the adverse profile rather than the therapeutic benefit in the relief of pain may be due to the blockade of brain voltage-gated sodium channels.

It will be appreciated that the invention may be modified within the scope of the appended claims.

1. (canceled)

2. (canceled)

3. A method for treating neuropathic pain comprising administering to a patient in need thereof an effective amount of at least one 5H-dibenz/b,f/azepine-5-carboxamide derivative selected from eslicarbazepine acetate, R-licarbazepine acetate and a mixture of eslicarbazepine acetate and R-licarbazepine acetate in any proportion in combination with:
a nonselective COX inhibitor selected from: acetylsalicylic acid, sodium salicylate, choline, magnesium trisalicylate, salsalate, diflunisaL sulfasalazine, olsalazine, and combinations thereof; acetaminophen; indometahcin, sulindac, and combinations thereof; tolmetin, diclofenac, ketorolac, and combinations thereof; ibuprofen, naproxen, flurbiprofen, ketoprofen, fenoprofen, oxaprozin, and combinations thereof; mephenamic acid, meclofenamic acid, and combinations thereof; piroxicam, meloxicam, and combinations thereof; and nabumetone;
a selective COX inhibitor selected from: rofecoxib, celecoxib, etoricoxib, parecoxib, valdecoxib, lumiracoxib, cimicoxib, and combinations thereof; etodolac; and nimesulide;
an opioid receptor agonist selected from: morphine, methadone, etorphine, codeine, hydrocodone, oxycodone, tramadol, levorphanol, meperidine, propoxyphene, fentanyl, sufentanil, alfentanil, remifentanii, and combinations thereof; and/or
an opioid receptor partial agonist selected from: pentazocine, butorphanol, buprenorphine and combinations thereof.

4. The method according to claim 3, wherein the 5H-dibenz/b,f/azepine-5-carboxamide derivative is eslicarbazepine acetate.

5. The method according to claim 3, wherein the 5H-dibenz/b,f/azepine-5-carboxamide derivative is R-licarbazepine acetate.

6. The method according to claim 3, wherein the 5H-dibenz/b,f/azepine-5-carboxamide derivative is a mixture of eslicarbazepine acetate and R-licarbazepine acetate in any proportion.

7. The method according to claim 6, wherein the 5H-dibenz/b,f/azepine-5-carboxamide derivative is the racemate of eslicarbazepine acetate and R-licarbazepine acetate.

8. The method according to claim 3, wherein the neuropathic pain is caused by trigeminal neuralgia, phantom pain, diabetic neuropathy or postherpetic neuralgia.

9. A method for treating at least one neurological disorder involving both motor impairment and neuropathic pain comprising administering to a patient in need thereof an effective amount of at least one 5H-dibenz/b,f/azepine-5-carboxamide derivative selected from eslicarbazepine acetate, R-licarbazepine acetate, a mixture of eslicarbazepine acetate and R-licarbazepine acetate in any proportion, S-licarbazepine, R-licarbazepine, a mixture of S-iicarbazepine and R-licarbazepine in any proportion, oxcarbazepine and carbamazepine.

10. The method according to claim 9, wherein the 5H-dibenz/b,f/azepine-5-carboxamide derivative is the racemate of eslicarbazepine acetate and R-licarbazepine acetate.

11. The method according to claim 9, wherein the 5H-dibenz/b,f/azepine-5-carboxamide derivative is the racemate of S-licarbazepine and R-licarbazepine.

12. A method for treating at least one neurological disorder involving both motor impairment and neuropathic pain comprising administering to a patient in need thereof an effective amount of at least one 5H-dibenz/b,f/azepine-5-carboxamide derivative selected from eslicarbazepine acetate, R-licarbazepine acetate, a mixture of eslicarbazepine acetate and R-licarbazepine acetate in any proportion, S-licarbazepine, R-licarbazepine, a mixture of S-licarbazepine and R-licarbazepine in any proportion, oxcarbazepine and carbamazepine in combination with:
a nonselective COX inhibitor selected from: acetylsalicylic acid, sodium salicylate, choline, magnesium trisalicylate, salsalate, diflunisal, sulfasalazine, olsalazine, and combinations thereof; acetaminophen; indometahcin, sulindac, and combinations thereof; tolmetin, diclofenac, ketorelac, and combinations thereof; ibuprofen, naproxen, flurbiprofen, ketoprofen, fenoprofen, oxaprozin, and combinations thereof; mephenamic acid, meclofenamic acid, and combinations thereof; piroxicam, meloxicam, and combinations thereof; and nabumetone;
a selective COX inhibitor selected from: rofecoxib, celecoxib, etoricoxib, parecoxib, valdecoxib, lumiracoxib, cimicoxib, and combinations thereof; etodolac; and nimesulide;
an opioid receptor agonist selected from: morphine, methadone, etorphine, codeine, hydrocodone, oxycodone, tramadol, levorphanol, meperidine, propoxyphene, fentanyl, sufentanil, alfentanil, remifentanil, and combinations thereof; and/or
an opioid receptor partial agonist selected from: pentazocine, butorphanol, buprenorphine and combinations thereof.

13. The method according to claim 12, wherein the 5H-dibenz/b,f/azepine-5-carboxamide derivative is eslicarbazepine acetate.

14. The method according to claim 12, wherein the 5H-dibenz/b,f/azepine-5-carboxamide derivative is R-licarbazepine acetate.

15. The method according to claim 12, wherein the 5H-dibenz/b,f/azepine-5-carboxamide derivative is a mixture of eslicarbazepine acetate and R-licarbazepine acetate in any proportion.

16. The method according to claim 15, wherein the 5H-dibenz/b,f/azepine-5-carboxamide derivative is the racemate of eslicarbazepine acetate and R-licarbazepine acetate.

17. The method according to claim 12, wherein the 5H-dibenz/b,f/azepine-5-carboxamide derivative is S-licarbazepine.

18. The method according to claim 12, wherein the 5H-dibenz/b,f/azepine-5-carboxamide derivative is R-licarbazepine.

19. The method according to claim 12, wherein the 5H-dibenz/b,f/azepine-5-carboxamide derivative is a mixture of S-licarbazepine and R-licarbazepine in any proportion.

20. The method according to claim 19, wherein the 5H-dibenz/b,f/azepine-5-carboxamide derivative is the racemate of S-licarbazepine and R-licarbazepine.

21. The method according to claim 12, wherein the 5H-dibenz/b,f/azepine-5-carboxamide derivative is oxcarbazepine.

22. The method according to claim 12, wherein the 5H-dibenz/b,f/azepine-5-carboxamide derivative is carbamazepine.

23. The method according to claim 9, wherein the at least one neurological disorder is selected from polyneuropathies; multiple sclerosis; Parkinson disease; CNS diseases with de-efferentiation caused by vascular, tumoral and inflammatory processes; motor neuron disease; progressive supranuclear palsy; multiple system atrophy; corticobasal degeneration; spinocerebellar ataxia; cervical myelopathy; spinal cord injury; and radicular avulsion.

24. (canceled)

25. (canceled)

26. The method according to claim 9, wherein the 5H-dibenz/b,f/azepine-5-carboxamide derivative is eslicarbazepine acetate.
